(19) United States
(12) Patent Application Publication
(10) Pub. No.: US 2021/0015722 A1
(43) Pub. Date: Jan. 21, 2021

(54) PROCESS FOR THE PREPARATION OF MICROCAPSULES

(71) Applicant: Firmenich SA, Geneva 8 (CH)

(72) Inventors: Florence VIGOUROUX ELIE, Geneva (CH); Sonia GODEFROY, Geneva (CH); Murray OSBORNE, Geneva (CH); Arnaud STRUILLOU, Geneva (CH)

(73) Assignee: Firmenich SA, Geneva 8 (CH)

(21) Appl. No.: 16/320,118

(22) PCT Filed: Jul. 26, 2017
(86) PCT No.: PCT/EP2017/068909

§ 371 Date: Jan. 24, 2019

(30) Foreign Application Priority Data
Jul. 27, 2016 (EP) 16181501.4

(51) Int. Cl. A61K 8/14; B29B 9/00; C11B 9/00

(52) U.S. Cl. CPC A61K 8/14; A61K 2800/412; C11B 9/00; B29B 9/00

(57) ABSTRACT

The present invention relates to a new process for the preparation of microcapsules. Microcapsules obtainable by said process are also an object of the invention. Perfuming compositions and consumer products comprising said capsules, in particular perfumed consumer products in the form of home care or personal care products, are also part of the invention.

### TECHNICAL FIELD

The present invention relates to a new process for the preparation of microcapsules. Microcapsules obtainable by said process are also an object of the invention. Perfuming compositions and consumer products comprising said microcapsules, in particular perfumed consumer products in the form of home care or personal care products, are also part of the invention.

### BACKGROUND OF THE INVENTION

One of the problems faced by the perfumery industry lies in the relatively rapid loss of olfactive benefit provided by odoriferous compounds due to their volatility, particularly that of “top-notes”. In order to tailor the release rates of volatiles, delivery systems such as microcapsules containing a perfume, are needed to protect and later release the core payload when triggered. A key requirement from the industry regarding these systems is to survive suspension in challenging bases without physically dissociating or degrading. This is referred to as performance in terms of stability for the delivery system. For instance, fragranced personal and household cleansers containing high levels of aggressive surfactant detergents are very challenging for the stability of microcapsules.

As described above, the performance in terms of stability represents an important requirement for perfume delivery systems. However, these delivery systems also must exhibit good performance in terms of perfume release, either during the wash/lathering phase (blooming) or on dry substrate (skin, hair, textile or home surface) after the wash.

Aminoplast microcapsules formed of a melamine-formaldehyde resin have been largely used to encapsulate hydrophobic actives, thus protecting said actives and providing their controlled release. However, capsules such as aminoplast ones suffer from stability problems when used in consumer products comprising surfactants, such as perfumery consumer products, especially after prolonged storage at elevated temperatures. In such products, even though the capsule wall remains intact, the encapsulated active tends to leak out of the capsule by diffusion through the wall due to the presence of surfactants that are able to solubilise the encapsulated active in the product base. The leakage phenomenon reduces the efficiency of the capsules to protect the active and provide its controlled release. Aminoplast microcapsules are also particularly suited for perfume release on dry substrate (skin, hair, textile or home surface) after the wash as they are very brittle once dry. However, they are not suited at all for perfume release during the wash/lathering phase (blooming) as they are extremely difficult to break when they are in water.

A variety of strategies have been described to improve the stability of oil core-based microcapsules. Cross-linking of capsule walls, with chemical groups such as polyamines and polyisocyanates, has been described as a way to improve stability of microcapsules. WO2011/154893 discloses for instance a process for the preparation of polyurea microcapsules using a combination of aromatic and aliphatic polyisocyanates in specific relative concentrations. Compared to aminoplast, polyurea-based microcapsules present the additional advantage of being free from melamine-formaldehyde. However, these polyurea-based microcapsules were designed and optimised to maximise perfume release upon rubbing after the wash, on dry substrate (skin, hair, fabrics). To this end, capsule breakage in the wash phase, upon lathering was minimised as it would reduce the amount of intact capsules left on substrate to be broken when dry in the post-wash phase.

There is therefore still a need to provide capsules free from melamine-formaldehyde which would be efficient in terms of blooming effect in the wash phase upon lathering and would retain some performance after rubbing on dry substrate, easy to manufacture and stable in challenging media such as surfactant-based consumer products.

### SUMMARY OF THE INVENTION

The process of the invention therefore provides a solution to the above-mentioned problems as it allows preparing microcapsules demonstrating a high performance notably in terms of blooming and stability.

As an example, a blooming fragrance can be defined by its blooming effect that characterizes the olfactive impact when any fragranced surfactant formulation is diluted during application lasting less than 90 seconds on average.

Therefore, a first object of the present invention is a process for the preparation of a core-shell microcapsule slurry comprising the steps of:
1) admixing a hydrophobic active ingredient with at least one polyisocyanate having at least three isocyanate functional groups to form an oil phase;
2) dissolving an ionic emulsifier in water to form a water phase;
3) adding the oil phase to the water phase to form an oil-in-water dispersion, wherein the mean droplet size is greater than 500 microns;
4) applying conditions sufficient to induce interfacial polymerisation and form core-shell microcapsules in form of a slurry, wherein the shell consists essentially of polymerised polyisocyanate having at least three isocyanate functional groups; and
5) Optionally, drying the capsule slurry to obtain dried microcapsules.

A second object of the present invention is a core-shell microcapsule slurry comprising microcapsules having
a) an oil-based core comprising an hydrophobic active ingredient;
b) a polymeric shell formed by interfacial polymerisation and consisting essentially of polymerised polyisocyanate formed from a polyisocyanate having at least three isocyanate functional groups in the presence of an ionic emulsifier;

characterized in that microcapsules have a size greater than 500 microns.

A third object of the present invention consists of a perfuming composition comprising
(i) microcapsules slurry or microcapsule powder defined in the invention, wherein the oil phase comprises a perfume;
(ii) at least one ingredient selected from the group consisting of a perfumery carrier and a perfuming co-ingredient; and
(iii) optionally a perfumery adjuvant.

Finally, a last object of the invention is a surfactant-based consumer product comprising microcapsule slurry, or microcapsule powder, or a perfuming composition as defined in the present invention.

### BRIEF DESCRIPTION OF THE FIGURES

FIG. 1 shows results from an evaluation of capsules in a shower gel composition, with the perfume intensity rated by a panel.

### DETAILED DESCRIPTION OF THE INVENTION

Unless stated otherwise, percentages (%) are meant to designate a percentage by weight of a composition.

By “perfume or flavour oil”, it is meant a single perfuming or flavouring compound or a mixture of several perfuming or flavouring compounds.

For the sake of clarity, by the expression “dispersion” in the present invention it is meant a system in which particles are dispersed in a continuous phase of a different composition and it specifically includes a suspension or an emulsion.

A “core-shell microcapsule”, or the similar, in the present invention it is meant that capsules have a particle size distribution in the micron range (e.g. a mean diameter (d(v, 0.5)) greater than 500 μm) and comprise an external solid oligomers-based shell or shell and an internal continuous oil phase enclosed by the external shell. In other words bodies like coacervates or extrudates (i.e. porous solid phases containing droplets of a liquid) are not part of the invention.

According to the invention, the wordings “mean diameter” or “mean size” are used indifferently.

Mean sizes were measured by a laser diffraction particle size analyzer.

According to the invention, the microcapsules are free from melamine-formaldehyde.

It has been found that microcapsules having a mean size greater than 500 microns with a good performance namely a right balance between stability in a surfactant-based product and blooming effect could be obtained without the need of more complex processes (such as coacervation).

A first object of the present invention is therefore a process for the preparation of a core-shell microcapsule slurry comprising the steps of:
1) admixing a hydrophobic active ingredient with at least one polyisocyanate having at least three isocyanate functional groups to form an oil phase;
2) dissolving an ionic emulsifier in water to form a water phase;
3) adding the oil phase to the water phase to form an oil-in-water dispersion, wherein the mean droplet size is greater than 500 microns; and
4) applying conditions sufficient to induce interfacial polymerisation and form core-shell microcapsules in form of a slurry, wherein the shell consists essentially of polymerised polyisocyanate having at least three isocyanate functional groups; and
5) Optionally, drying the capsule slurry to obtain dried microcapsules.

### Step 1 : Providing an Oil Phase

In the first step of the process, a hydrophobic active ingredient is admixed with at least one polyisocyanate having at least three isocyanate functional groups to form an oil phase.

### Hydrophobic Active Ingredient

By “hydrophophic active ingredient”, it is meant any active ingredient—single ingredient or a mixture of ingredients—which forms a two-phases solution in water.

Hydrophobic active ingredients are preferably chosen from the group consisting of flavor, flavor ingredients, perfume, perfume ingredients, nutraceuticals, cosmetics, insect control agents, biocide actives and mixtures thereof.

The nature and type of the insect control agents present in the oil phase do not warrant a more detailed description here, which in any case would not be exhaustive, the skilled person being able to select them on the basis of its general knowledge and according to intended use or application.

Examples of such insect control agents are birch, DEET (N,N-diethyl-m-toluamide), essential oil of the lemon eucalyptus (Corymbia citriodora) and its active compound p-menthane-3,8-diol(PMD), icaridin (hydroxyethyl isobutyl piperidine carboxylate), Nepelactone, Citronella oil, Neem oil, Bog Myrtle (Myrica Gale), Dimethyl carbate, Tricyclodecenyl allyl ether, IR3535 (3-[N-Butyl-N-acetyl]-aminopropionic acid, ethyl ester, Ethylhexanediol, Dimethyl phthalate, Metofluthrin, Indalone, SS220, anthranilate-based insect repellents, and mixtures thereof.

According to a particular embodiment, the hydrophobic-active ingredient comprises a mixture of a perfume with another ingredient selected from the group consisting of nutraceuticals, cosmetics, insect control agents and biocide actives.

According to a particular embodiment, the hydrophobic active ingredient comprises a perfume.

According to a particular embodiment, the hydrophobic active ingredient consists of a perfume.

By “perfume oil” (or also “perfume”) what is meant here is an ingredient or composition that is a liquid at about 20° C. According to any one of the above embodiments said perfume oil can be a perfuming ingredient alone or a mixture of ingredients in the form of a perfuming composition. As a “perfuming ingredient” it is meant here a compound, which is used for the primary purpose of conferring or modulating an odour. In other words such an ingredient, to be considered as being a perfuming one, must be recognized by a person skilled in the art as being able to at least impart or modify in a positive or pleasant way the odor of a composition, and not just as having an odor. For the purpose of the present invention, perfume oil also includes combination of perfuming ingredients with substances which together improve, enhance or modify the delivery of the perfuming ingredients, such as perfume precursors, emulsions or dispersions, as well as combinations which impart an additional benefit beyond that of modifying or imparting an odor, such as long-lasting, blooming, malodour counteraction, antimicrobial effect, microbial stability, insect control.

The nature and type of the perfuming ingredients present in the oil phase do not warrant a more detailed description here, which in any case would not be exhaustive, the skilled person being able to select them on the basis of its general knowledge and according to intended use or application and the desired organoleptic effect. In general terms, these perfuming ingredients belong to chemical classes as varied as alcohols, aldehydes, ketones, esters, ethers, acetates, nitriles, terpenoids, nitrogenous or sulphurous heterocyclic compounds and essential oils, and said perfuming co-ingredients can be of natural or synthetic origin. Many of these co-ingredients are in any case listed in reference texts such as the book by S. Arctander, Perfume and Flavor Chemicals, 1969, Montclair, N.J., USA, or its more recent versions, or in other works of a similar nature, as well as in the abundant patent literature in the field of perfumery. It is also understood that said ingredients may also be compounds known to release in a controlled manner various types of perfuming compounds.

The perfuming ingredients may be dissolved in a solvent of current use in the perfume industry. The solvent is preferably not an alcohol. Examples of such solvents are diethyl phthalate, isopropyl myristate, Abalyn® (rosin resins, available from Eastman), benzyl benzoate, ethyl citrate, limonene or other terpenes, or isoparaffins. Preferably, the solvent is very hydrophobic and highly sterically hindered, like for example Abalyn® or benzyl benzoate. Preferably the perfume comprises less than 30% of solvent. More preferably the perfume comprises less than 20% and even more preferably less than 10% of solvent, all these percentages being defined by weight relative to the total weight of the perfume. Most preferably, the perfume is essentially free of solvent.

According to an embodiment, the hydrophobic active ingredient represents between 20 to 50% by weight relative to the total weight of the dispersion as obtained after step 3).

Polyisocyanate having at Least Three Isocyanate Functional Groups

Suitable polyisocyanates used according to the invention include aromatic polyisocyanate, aliphatic polyisocyanate and mixtures thereof. According to the invention, the oil phase comprises at least one polyisocyanate having at least 3 but may comprise up to 6, or even only 4, isocyanate functional groups.

According to a particular embodiment, a triisocyanate (3 isocyanate functional groups) is used.

According to an embodiment, a mixture of a diisocyanate (2 isocyanate functional groups) with a triisocyanate (3 isocyanate functional groups) is used.

According to a particular embodiment, the oil phase is essentially free from diisocyanate.

By “essentially free from diisocyanate” it is meant that the water phase does not contain an amount of diisocyanate susceptible of reacting in a way that would substantially modify the nature of the capsule shell.

According to an embodiment, the oil phase is completely free from diisocyanate.

According to one embodiment, said polyisocyanate is an aromatic polyisocyanate.

The term “aromatic polyisocyanate” is meant here as encompassing any polyisocyanate comprising an aromatic moiety. Preferably, it comprises a phenyl, a toluyl, a xylyl, a naphthyl or a diphenyl moiety, more preferably a toluyl or a xylyl moiety. Preferred aromatic polyisocyanates are biurets, polyisocyanurates and trimethylol propane adducts of diisocyanates, more preferably comprising one of the above-cited specific aromatic moieties. More preferably, the aromatic polyisocyanate is a polyisocyanurate of toluene diisocyanate (commercially available from Bayer under the tradename Desmodur® RC), a trimethylol propane-adduct of toluene diisocyanate (commercially available from Bayer under the tradename Desmodur® L75), a trimethylol propane-adduct of xylylene diisocyanate (commercially available from Mitsui Chemicals under the tradename Takenate® D-110N). In a most preferred embodiment, the aromatic polyisocyanate is a trimethylol propane-adduct of xylylene diisocyanate.

According to another embodiment, said polyisocyanate is an aliphatic polyisocyanate. The term “aliphatic polyisocyanate” is defined as a polyisocyanate which does not comprise any aromatic moiety. Preferred aliphatic polyisocyanates are a trimer of hexamethylene diisocyanate, a trimer of isophorone diisocyanate, a trimethylol propane-adduct of hexamethylene diisocyanate (available from Mitsui Chemicals) or a biuret of hexamethylene diisocyanate (commercially available from Bayer under the tradename Desmodur® N 100), among which a biuret of hexamethylene diisocyanate is even more preferred.

According to another embodiment, said at least one polyisocyanate is in the form of a mixture of at least one aliphatic polyisocyanate and of at least one aromatic polyisocyanate, both comprising at least two or three isocyanate functional groups, such as a mixture of a biuret of hexamethylene diisocyanate with a trimethylol propane-adduct of xylylene diisocyanate, a mixture of a biuret of hexamethylene diisocyanate with a polyisocyanurate of toluene diisocyanate and a mixture of a biuret of hexamethylene diisocyanate with a trimethylol propane-adduct of toluene diisocyanate. Most preferably, it is a mixture of a biuret of hexamethylene diisocyanate with a trimethylol propane-adduct of xylylene diisocyanate. Preferably, when used as a mixture the molar ratio between the aliphatic polyisocyanate and the aromatic polyisocyanate is ranging from 80:20 to 10:90.

The at least one polyisocyanate used in the process of the invention is present in amounts representing from 1 to 15 wt %, preferably from 1.5 to 12 wt %, more preferably from 2 to 8 wt % and even more preferably from 2 to 6 wt % of the oil phase.

According to a particular embodiment, the oil phase essentially consists of the polyisocyanate with at least 3 isocyanate functional groups, and the perfume or flavor oil.

Step 2: Dissolving an Ionic Emulsifier in Water to form a Water Phase

The polymeric shell of the microcapsule according to the present invention is formed by interfacial polymerization in the presence of an ionic emulsifier.

According the invention, an ionic emulsifier is solubilized in water to form a water phase, preferably at pH>5.

According to an embodiment, the ionic emulsifier is chosen in the group consisting of gum Arabic, carboxymethyl cellulose, soy protein, sodium caseinate, gelatin, bovine serum albumin, sugar beet pectin, hydrolyzed soy protein, hydrolyzed sericin, Pseudocollagen, Biopolymer SA-N (INCI name: Hyaluronic Acid (and) Serum Albumen (and) Dextran Sulfate), Pentacare-NA PF (Hydrolyzed Wheat Gluten (and) Ceratonia Siliqua (Carob) Gum (and) Aqua (and) Sodium Dextran Sulfate (and) Bis-Hydroxyethyl Tromethamine (and) Phenoxyethanol (and) Ethylhexylglycerin), co-polymers of acrylamide and acrylic acid (such as Alcapsol® 144 from Ciba), e.g. acid/acrylamide copolymers produced from monomer mixture of acrylic acid and acrylamide wherein the acrylic acid content is in the range of from 30 to 70%,), acrylic co-polymers bearing a sulfonate group (such as sodium polystyrene sulfonate), and co-polymers of vinyl ethers and maleic anhydride (once hydrolysed) and mixtures thereof.

According to a preferred embodiment, the ionic emulsifier is chosen in the group consisting of gum Arabic, carboxymethyl cellulose, sodium caseinate, sugar beet pectin, co-polymers of acrylamide and acrylic acid and mixtures thereof.

According to any one of the above embodiments of the present invention, the dispersion comprises between about 0.1% and 5% w/w of the emulsifier, percentage being expressed on a w/w basis relative to the total weight of the dispersion as obtained after step 3). In still another aspect of the invention, the dispersion comprises between about 0.1% and 2% w/w of the emulsifier. In still another aspect of the invention, the dispersion comprises between about 0.1% and 1% w/w of the emulsifier.

According to a first embodiment, capsules according to the present invention are polyurea-based capsules. According to a particular embodiment, interfacial polymerization is induced by addition of a polyamine reactant. Preferably, the reactant is selected from the group consisting of water soluble guanidine salts, tris-(2-aminoethyl)amine, N,N,N′,N′-tetrakis(3-aminopropyl)-1,4-butanediamine and guanazole to form a polyurea wall with the polyisocyanate.

According to particular embodiment, polyurea-based capsules are formed in absence of a substantial amount of added polyamine reactant, and result only from the autopolymerization of the at least one polyisocyanate, preferably in the presence of a catalyst.

According to this particular embodiment, “in absence of a substantial amount of added polyamine reactant” means that the amount of amine or polyamine added has to be sufficiently low so as not to be able to significantly change the properties of the microcapsule shell if it reacts with the polyisocyanate. Typically, the amount of amine functionalities that can be added is less than 50% molar, preferably less than 25% molar, most preferably less than 10% molar of the amount of isocyanate functionalities.

According to a particular embodiment, polyurea-based capsules are formed in absence of added polyamine reactant.

According to an embodiment, no substantial amount of other water-soluble reactant than amine or polyamine susceptible to polymerize with the polyisocyanate is added at any stage of the process, said water-soluble reactant being chosen in the group consisting of polyols, thiols, ureas, urethanes and mixtures thereof.

Thus, according to an embodiment, polyurea-based capsules are formed in absence of a reactant chosen in the group consisting of amine, polyamine, polyols, thiols, ureas, urethanes and mixtures thereof. According to a third embodiment, capsules according to the present invention are polyurethane-based capsules. According to this particular embodiment, interfacial polymerization is induced by addition of a polyol reactant. Preferably the reactant is selected from the group consisting of monomeric and polymeric polyols with multiple hydroxyl groups available for reaction and mixtures thereof.

According to a fourth embodiment, capsules according to the present invention are polyurea/polyurethane based. In that case interfacial polymerization is induced by addition of a mixture of the reactant mentioned under precedent first and second embodiments. Additionally, crosslinkers with both amino groups and hydroxyl groups can be used to generate polyurea/polyurethane materials. Furthermore, polyisocyanates with both urea and urethane functionalities can be used to generate polyurea/polyurethane materials.

According to a fifth embodiment, capsules according to the present invention are organic-inorganic hybrid capsules. According to this particular embodiment, an orthosilicate, a silane or a combination of silanes can be added from the oil phase or the water phase to form a hybridized inorganic/organic membrane or surface coating. Silanes can be suspended in the oil phase to silicify the inner membrane, or can be added post-emulsification to form a silicified shell around the burgeoning polymeric capsule membrane. Inside-out and outside-in sol gel polymerization can occur by forming and hardening 3D siloxane bonds inside or outside the polymer membrane via condensation of alkoxide in or on the emulsion droplets.

Process conditions for interfacial polymerization do not need further description here as they are well known to a skilled person in the art.

### Step 3: Admixing the Oil Phase and the Water Phase to Form a Dispersion

In the next step of the process of the invention, the oil phase is then added to the water phase to form a dispersion.

According to the invention, the mean droplet size of the oil-in-water emulsion is greater than 500 μm.

The person skilled in the art will be able to select a suitable stirring speed to achieve a mean droplet size greater than 500 microns.

According to an embodiment, the mean droplet size of the oil-in-water emulsion is comprised between greater than 500 microns and less than 3000 microns, preferably greater than 500 microns and less than 2000 microns, more preferably greater than 500 microns and less than 1500 microns.

### Step 4: Curing Step

This is followed by a curing step 4) which allows to end up with microcapsules in the form of a slurry or liquid dispersion. According to a preferred embodiment, said step is performed at a temperature comprised between 50 and 130° C., possibly under pressure, for 15 minutes to 8 hours. More preferably it is performed at between 50 and 90° C. for between 30 minutes and 4 hours. Most preferably it is performed between 75 and 90° C. for between 1 and 4 hours.

### Optional Steps

### Outer Coating

According to a particular embodiment of the invention, at the end of step 4) one may also add to the invention's slurry a polymer selected from the group consisting of a non-ionic polysaccharide, a cationic polymer and mixtures thereof to form an outer coating to the microcapsule. Such coating will help drive capsule deposition and retention on substrate during the wash process so that a significant part of the capsules which have not been broken in the wash phase/upon lathering would transfer to the substrate (skin, hair fabrics) and be available for perfume release when the capsules are broken upon rubbing after drying.

According to a particular embodiment, the coating consists of a cationic coating.

Non-ionic polysaccharide polymers are well known to a person skilled in the art. Preferred non-ionic polysaccharides are selected from the group consisting of locust bean gum, xyloglucan, guar gum, hydroxypropyl guar, hydroxypropyl cellulose and hydroxypropyl methyl cellulose.

Cationic polymers are also well known to a person skilled in the art. Preferred cationic polymers have cationic charge densities of at least 0.5 meq/g, more preferably at least about 1.5 meq/g, but also preferably less than about 7 meq/g, more preferably less than about 6.2 meq/g. The cationic charge density of the cationic polymers may be determined by the Kjeldahl method as described in the US Pharmacopoeia under chemical tests for Nitrogen determination. The preferred cationic polymers are chosen from those that contain units comprising primary, secondary, tertiary and/or quaternary amine groups that can either form part of the main polymer chain or can be borne by a side substituent directly connected thereto. The weight average (Mw) molecular weight of the cationic polymer is preferably between 10,000 and 2M Dalton, more preferably between 50,000 and 3.5M Dalton.

According to a particular embodiment, one will use cationic polymers based on acrylamide, methacrylamide, N-vinylpyrrolidone, quaternized N,N-dimethylaminomethacrylate, diallyldimethylammonium chloride, quaternized vinylimidazole (3-methyl-1-vinyl-1H-imidazol-3-ium chloride), vinylpyrrolidone, acrylamidopropyltrimonium chloride, cassia hydroxypropyltrimonium chloride, guar hydroxypropyltrimonium chloride or polygalactomannan 2-hydroxypropyltrimethylammonium chloride ether, starch hydroxypropyltrimonium chloride and cellulose hydroxypropyltrimonium chloride. Preferably copolymers shall be selected from the group consisting of polyquaternium-5, polyquaternium-6, polyquaternium-7, polyquaternium10, polyquaternium-11, polyquaternium-16, polyquaternium-22, polyquaternium-28, polyquaternium-43, polyquaternium-44, polyquaternium-46, cassia hydroxypropyltrimonium chloride, guar hydroxypropyltrimonium chloride or polygalactomannan 2-hydroxypropyltrimethylammonium chloride ether, starch hydroxypropyltrimonium chloride and cellulose hydroxypropyltrimonium chloride

As specific examples of commercially available products, one may cite Salcare® SC60 (cationic copolymer of acrylamidopropyltrimonium chloride and acrylamide, origin: BASF) or Luviquat®, such as the PQ 11N, FC 550 or Style (polyquaternium-11 to 68 or quaternized copolymers of vinylpyrrolidone origin: BASF), or also the Jaguar® (C135 or C17, origin Rhodia). According to any one of the above embodiments of the invention, there is added an amount of polymer described above comprised between about 0% and 5% w/w, or even between about 0.1% and 2% w/w, percentage being expressed on a w/w basis relative to the total weight of the slurry as obtained after step 4). It is clearly understood by a person skilled in the art that only part of said added polymers will be incorporated into/deposited on the microcapsule shell.

### Drying

According to an embodiment, the slurry obtained by the process described above can be submitted to a drying. In particular, the person skilled in the art will be able to select a suitable method for drying notably according to the size of the microcapsules.

A microcapsule slurry or a microcapsule powder obtainable by a process as defined in any of the above-embodiment is another object of the invention.

Another object of the present invention is a core-shell microcapsule slurry obtainable by the process disclosed above.

Another object of the present invention is a core-shell microcapsule slurry comprising microcapsules having

a) an oil-based core comprising an hydrophobic active ingredient;

b) a polymeric shell formed by interfacial polymerisation consisting essentially of polymerised polyisocyanate formed from a polyisocyanate having at least three isocyanate functional groups in the presence of an ionic emulsifier;

characterized in that microcapsules have a mean size greater than 500 microns.

Technical features described for the process of the invention also apply for the core-shell microcapsule slurry mentioned above.

The capsules of the invention show very good performance in terms of stability in challenging medium, good mechanical properties which translate into good odor performance as well as good blooming properties. In this regard it has to be mentioned that although ideal situation would be one where microcapsules show best stability, i.e. lowest perfume leakage in application combined with best odor performance, i.e. perfume intensity in application both before rubbing and after rubbing, different scenarios can be very interesting depending on the application and slightly less stable capsules with higher odor performance can be very useful and so could more stable capsules with slightly lower odor performance. The capsules of the invention have a profile perfume leakage/odor performance that varies depending on the proportion of polyisocyanate and the nature of the perfume oil. A skilled person in the art is capable of choosing the best balance depending on the needs in application. The capsules according to the invention present the additional advantage of being free from melamine-formaldehyde.

Another object of the present invention is a perfuming composition comprising:

(i) Perfume microcapsule slurry or microcapsule powder as defined above;

(ii) At least one ingredient selected from the group consisting of a perfumery carrier, a perfumery co-ingredient and mixtures thereof;

(iii) Optionally at least one perfumery adjuvant.

As liquid perfumery carrier one may cite, as non-limiting examples, an emulsifying system, i.e. a solvent and a surfactant system, or a solvent commonly used in perfumery. A detailed description of the nature and type of solvents commonly used in perfumery cannot be exhaustive. However, one can cite as non-limiting examples solvents such as dipropyleneglycol, diethyl phthalate, isopropyl myristate, benzyl benzoate, 2-(2-ethoxyethoxy)-1-ethanol or ethyl citrate, which are the most commonly used. For the compositions which comprise both a perfumery carrier and a perfumery co-ingredient, other suitable perfumery carriers than those previously specified, can be also ethanol, water/ethanol mixtures, limonene or other terpenes, isoparaffins such as those known under the trademark Isopar® (origin: Exxon Chemical) or glycol ethers and glycol ether esters such as those known under the trademark Dowanol® (origin: Dow Chemical Company). By “perfumery co-ingredient” it is meant here a compound, which is used in a perfuming preparation or a composition to impart a hedonic effect and which is not a microcapsule as defined above. In other words such a co-ingredient, to be considered as being a perfuming one, must be recognized by a person skilled in the art as being able to impart or modify in a positive or pleasant way the odor of a composition, and not just as having an odor.

The nature and type of the perfuming co-ingredients present in the perfuming composition do not warrant a more detailed description here, which in any case would not be exhaustive, the skilled person being able to select them on the basis of his general knowledge and according to the intended use or application and the desired organoleptic effect. In general terms, these perfuming co-ingredients belong to chemical classes as varied as alcohols, lactones, aldehydes, ketones, esters, ethers, acetates, nitriles, terpenoids, nitrogenous or sulphurous heterocyclic compounds and essential oils, and said perfuming co-ingredients can be of natural or synthetic origin. Many of these co-ingredients are in any case listed in reference texts such as the book by S. Arctander, Perfume and Flavor Chemicals, 1969, Montclair, N.J., USA, or its more recent versions, or in other works of a similar nature, as well as in the abundant patent literature in the field of perfumery. It is also understood that said co-ingredients may also be compounds known to release in a controlled manner various types of perfuming compounds.

By “perfumery adjuvant” we mean here an ingredient capable of imparting additional added benefit such as a color, a particular light resistance, chemical stability, etc. A detailed description of the nature and type of adjuvant commonly used in perfuming bases cannot be exhaustive, but it has to be mentioned that said ingredients are well known to a person skilled in the art.

Preferably, the perfuming composition according to the invention comprises between 0.05 to 30%, preferably between 0.1 and 30% by weight of microcapsules as defined above.

The invention's microcapsules can advantageously be used in many application fields and used in consumer products. Microcapsules can be used in liquid form applicable to liquid consumer products as well as in powder form, applicable to powder consumer products.

Another object of the present invention is a liquid consumer product comprising:
a) from 2 to 65% by weight, relative to the total weight of the consumer product, of at least one surfactant;
b) water or a water-miscible hydrophilic organic solvent; and
c) microcapsules as defined above,
d) optionally non-encapsulated perfume.

A powder consumer product comprising
(a) from 2 to 65% by weight, relative to the total weight of the consumer product, of at least one surfactant;
(b) microcapsules as defined above.
(c) optionally perfume powder that is different from the microcapsules defined above is also an object according to the present invention.

In the case of microcapsules including a perfume oil-based core, the products of the invention, can in particular be of used in perfumed consumer products such as product belonging to fine fragrance or “functional” perfumery. Functional perfumery includes in particular personal-care products including hair-care, body cleansing, skin care, hygiene-care as well as home-care products including laundry care and air care. Consequently, another object of the present invention consists of a perfumed consumer product comprising as a perfuming ingredient, the microcapsules defined above or a perfuming composition as defined above. The perfume element of said consumer product can be a combination of perfume microcapsules as defined above and free or non-encapsulated perfume, as well as other types of perfume microcapsule than those here-disclosed.

In particular a liquid consumer product comprising:
a) from 2 to 65% by weight, relative to the total weight of the consumer product, of at least one surfactant;
b) water or a water-miscible hydrophilic organic solvent; and
c) a perfuming composition as defined above is another object of the invention.

Also a powder consumer product comprising:
(a) from 2 to 65% by weight, relative to the total weight of the consumer product, of at least one surfactant; and
(b) a perfuming composition as defined above is part of the invention.

The invention's microcapsules can therefore be added as such or as part of an invention's perfuming composition in a perfumed consumer product.

For the sake of clarity, it has to be mentioned that, by “perfumed consumer product” it is meant a consumer product which is expected to deliver among different benefits a perfuming effect to the surface to which it is applied (e.g. skin, hair, textile, paper, or home surface) or in the air (air-freshener, deodorizer etc). In other words, a perfumed consumer product according to the invention is a manufactured product which comprises a functional formulation also referred to as “base”, together with benefit agents, among which an effective amount of microcapsules according to the invention.

The nature and type of the other constituents of the perfumed consumer product do not warrant a more detailed description here, which in any case would not be exhaustive, the skilled person being able to select them on the basis of his general knowledge and according to the nature and the desired effect of said product. Base formulations of consumer products in which the microcapsules of the invention can be incorporated can be found in the abundant literature relative to such products. These formulations do not warrant a detailed description here which would in any case not be exhaustive. The person skilled in the art of formulating such consumer products is perfectly able to select the suitable components on the basis of his general knowledge and of the available literature.

Non-limiting examples of suitable perfumery consumer product can be a perfume, such as a fine perfume, a cologne or an after-shave lotion; a fabric care product, such as a liquid or solid detergent, tablets and pods, a fabric softener, a dryer sheet, a fabric refresher, an ironing water, or a bleach; a body-care product, such as a hair care product (e.g. a shampoo, hair conditioner, a colouring preparation or a hair spray), a cosmetic preparation (e.g. a vanishing cream, body lotion or a deodorant or antiperspirant), or a skin-care product (e.g. a perfumed soap, shower or bath mousse, body wash, oil or gel, bath salts, or a hygiene product); an air care product, such as an air freshener or a “ready to use” powdered air freshener; or a home care product, such all-purpose cleaners, liquid or power or tablet dishwashing products, toilet cleaners or products for cleaning various surfaces, for example sprays & wipes intended for the treatment/refreshment of textiles or hard surfaces (floors, tiles, stone-floors etc.), a hygiene product such as sanitary napkins, diapers, toilet paper. According to a particular embodiment, the consumer product is selected from the group consisting of a shampoo, a shower gel, a rinse-off conditioner, a soap bar, a powder or a liquid detergent, a fabric softener and a floor cleaner.

Preferably, the consumer product comprises from 0.05 wt %, preferably from 0.1 to 15wt %, more preferably between 0.2 and 5wt % of the microcapsules of the present invention, these percentages being defined by weight relative to the total weight of the consumer product. Of course the above concentrations may be adapted according to the olfactive effect desired in each product.

The capsules of the invention have demonstrated an improved blooming effect compared to capsules having a small size.

The invention will now be further described by way of examples. It will be appreciated that the invention as claimed is not intended to be limited in any way by these examples.

### EXAMPLES

### Example 1

Preparation of Capsules According to the Invention having a Size of 600 microns (Capsule A)

The oil phase was prepared by admixing a 2.15 g of polyisocyanate (trimethylol propane adduct of xylylene diisocyanate, Takenate® D-110N, origin and trademark from Mitsui Chemicals) with 19.55 g of perfume oil A (see table 1).

The aqueous phase was prepared by dissolving 0.98 g of gum arabic in 76.74 g of water. The emulsion was prepared by dispersing the perfume/polyisocyanate premix oil in the aqueous phase with the stirrer at 230 rpm to achieve a droplet size of 600 microns. The temperature was raised to 70° C. and was kept at 70° C. for 1h30 to allow the curing of the capsules. At this point, capsules were formed, cross-linked and stable. The mixture was left to cool down to room temperature.

The size distribution of the capsules was controlled by Optical Microscopy and Light Scattering (Mastersizer 3000, Malvern).

### Example 2 (Comparative)

Preparation of Capsules Comprising a Cationic Coating having a size of 40 microns (Capsule B)

The oil phase was prepared by admixing 15.35 g of polyisocyanate (trimethylol propane adduct of xylylene diisocyanate, Takenate® D-110N, origin and trademark from Mitsui Chemicals) with 307.0 g of perfume oil A (see table 1 below).

The aqueous phase was prepared by dissolving 7.40 g of Gum Arabic (origin and trademark from Alland & Robert) in 501.07 g of water. The emulsion was prepared by dispersing the perfume/polyisocyanate premix oil in the aqueous phase with the stirrer at 1050 rpm to achieve a droplet size of 40 microns.

The temperature was raised to 80° C. and was kept at 80° C. for 2h to allow the curing of the capsules. At this point, capsules were formed, cross-linked and stable. A 3% Salcare® SC60 (acrylamidepropyltrimonium chloride/acrylamide copolymer) solution in water was then added into the mixture at 80° C. and was allowed to react for 1 hour at 80° C. The mixture was left to cool down to room temperature.

After encapsulation and use of the Takenate® D-110N to produce the capsule wall, the residual level of unreacted polyisocyanate in the perfume oil was very low and therefore the internal core of the capsule was essentially made of the perfume oil.

The size distribution of the capsules is 40 microns and was controlled by Optical Microscopy and Light Scattering (Mastersizer 3000, Malvern).

### Example 3 (Comparative)

Preparation of Anionic Capsules having a Size of 35 Microns (Capsule C)

The oil phase was prepared by admixing 18.50 g of polyisocyanate (trimethylol propane adduct of xylylene diisocyanate, Takenate® D-110N, origin and trademark from Mitsui Chemicals) with 368.91 g of perfume oil A (see table 1).

The aqueous phase was prepared by dissolving 8.89 g of Gum Arabic (origin and trademark from Alland & Robert) in 602.1 g of water. The emulsion was prepared by dispersing the perfume/polyisocyanate premix oil in the aqueous phase with the stirrer at 1050 rpm to achieve a droplet size of 35 microns.

The temperature was raised to 80° C. and was kept at 80° C. for 2 h to allow the curing of the capsules. At this point, capsules were formed, cross-linked and stable. The mixture was left to cool down to room temperature.

After encapsulation and use of the Takenate® D-110N to produce the capsule wall, the residual level of unreacted polyisocyanate in the perfume oil was very low and therefore the internal core of the capsule was essentially made of the perfume oil.

The size distribution of the capsules is 35 microns and was controlled by Optical Microscopy and Light Scattering (Mastersizer 3000, Malvern).

TABLE 1
Composition of Perfume A

| Raw materials | % in formula |
|---|---|
| STIRRALLYL ACETATE | 4.5% |
| BENZYL ACETATE | 0.9% |
| ALDEHYDE C10 | 2.1% |
| HEXYLCINNAMIC | 14.3% |
| ALDEHYDE | |
| ALLYL CAPROATE | 0.7% |
| Ethyl 2-methyl-pentanoate | 0.9% |
| BENZYL BENZOATE | 35.3% |
| CITRONELLYL NITRILE | 1.8% |
| CORANOL ¹⁾ | 5.4% |
| DIHYDROMYRCENOL | 5.4% |
| FRUCTALATE ® ²⁾ | 5.4% |
| HEDIONE ® ³⁾ | 14.9% |
| LIMONENE | 2.4% |
| LINALOOL | 1.0% |
| METHYL | 1.5% |
| METHYLANTHRANILATE | |
| PARACYMENE | 1.7% |
| RHUBOFLOR | 0.7% |
| TERPINENE G | 0.6% |
| 2,4-Dimethyl-3-cyclohexene- | 0.6% |
| 1-carbaldehyde | |
| TOTAL | 100% |

¹⁾ 4-cyclohexyl-2-methyl-2-butanol; origin and Trademark from Firmenich SA, Geneva, Switzerland
²⁾ 1,4-cyclohexane dicarboxylate de diéthyle; origin and Trademark from Firmenich SA, Geneva, Switzerland
³⁾ Methyl dihydrojasmonate; origin and Trademark from Firmenich SA, Geneva, Switzerland

### Example 4 (Comparative)

Olfactive Performance of Capsules According to the Invention (capsule A) and Comparison with Capsules (Capsules C—not Part of the Invention)

Capsules were dispersed into a structured shower-gel base having the following composition: 50% deionized water, 5% thickener (acrylates/beheneth-25 methacrylate copolymer, available from Lubrizol), 43% surfactants (sodium pareth sulfate and cocamidopropyl betaine), 0.5% preservative (sodium benzoate); sodium hydroxide and citric acid are used to adjust the pH value.

Encapsulated perfume concentration in shower-gel base is equivalent to 0.20%.
Protocol for evaluation: by 8 expert panelists
   1) 1 ml of each shower-gels containing capsules is applied on the forearm:
   2) Evaluation of the fragrance intensity on a scale from 1 to 7
   3) Lathering step during few seconds. New evaluation of the fragrance intensity on a scale from 1 to 7
Evaluation scale: (fragrance intensity): 1=no fragrance odor; 2=just detectable; 3=weak; 4=moderate; 5=slightly strong; 6=intense; 7=very intense.

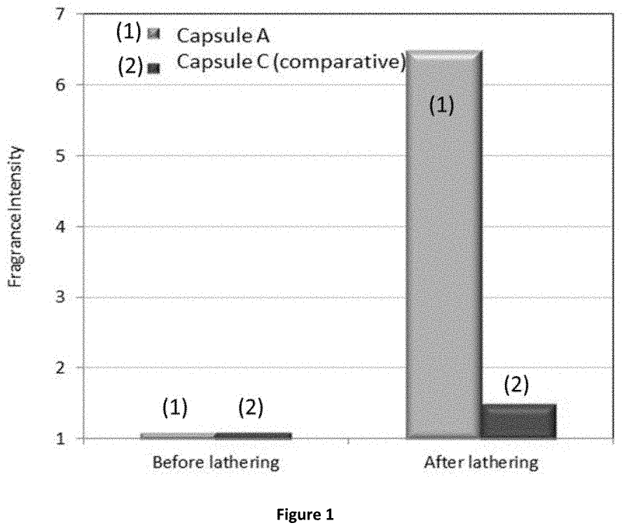

Results
Results are shown on FIG. 1.
One can conclude that:
1) Before lathering, the fragrance intensity is low for both capsules A and C underlying that there is few amount of perfume oil leaking out of the capsules
2) However, after lathering, the olfactive performance of capsules A according to the invention is much better than capsules C underlying that large particle size is needed to break enough capsules upon lathering to have a strongly perceivable perfume release.

1. A process for the preparation of a core-shell microcapsule slurry comprising the steps of:
1) admixing a hydrophobic active ingredient with at least one polyisocyanate having at least three isocyanate functional groups to form an oil phase;
2) dissolving an ionic emulsifier in water to form a water phase;
3) adding the oil phase to the water phase to form an oil-in-water dispersion, wherein the mean droplet size is greater than 500 microns;
4) applying conditions sufficient to induce interfacial polymerisation and form core-shell microcapsules in form of a slurry, wherein the shell consists essentially of polymerised polyisocyanate having at least three isocyanate functional groups; and
5) optionally, drying the capsule slurry to obtain dried microcapsules.

2. The process according to claim 1, wherein the mean droplet size of the oil-in-water dispersion is comprised between greater than 500 microns and less than 3000 microns.

3. The process according to claim 1, wherein the ionic emulsifier is chosen in the group consisting of gum Arabic, soy protein, sodium caseinate, gelatin, bovine serum albumin, sugar beet pectin, hydrolyzed soy protein, hydrolyzed sericin, Pseudocollagen, Biopolymer SA-N, Pentacare-NA PF, co-polymers of acrylamide and acrylic acid, acrylic co-polymers bearing a sulfonate group, co-polymers of vinyl ethers and maleic anhydride and mixtures thereof.

4. The process according to claim 1, further comprising the step of adding a polymer selected from the group consisting of a non-ionic polysaccharide, a cationic polymer and mixtures thereof to form outer coatings to the microcapsules after step 4).

5. The process according to claim 1, wherein under step 2) a polyamine and/or a polyol is added to the water phase.

6. The process according to claim 1, wherein the microcapsules are polyurea-based and wherein the process is performed in the absence of a substantial amount of added amine or polyamine.

7. The process according to claim 1, wherein the hydrophobic active ingredient represents between 20 to 50% by weight relative to the total weight of the dispersion.

8. The process according to claim 1, wherein the emulsifier is used in an amount comprised between 0.1 and 5 wt % relative to the total weight of the dispersion.

9. The process according to claim 1, wherein hydrophobic active ingredient is chosen in the group consisting of a perfume, flavor, nutraceuticals, cosmetics, insect control agents, biocide actives and mixtures thereof, preferably a perfume or flavour.

10. A core-shell microcapsule slurry obtainable by the process as defined in claim 1.

11. A core-shell microcapsule slurry comprising microcapsules having
a) an oil-based core comprising a hydrophobic active ingredient;
b) a polymeric shell formed by interfacial polymerisation and consisting essentially of polymerised polyisocyanate formed from a polyisocyanate having at least three isocyanate functional groups in the presence of an ionic emulsifier; characterized in that microcapsules have a size greater than 500 microns.

12. A core-shell microcapsules powder obtained by drying the microcapsule slurry according to claim 10.

13. A perfuming composition comprising:
(i) a microcapsules slurry as defined in claim 10, wherein the hydrophobic active ingredient comprises a perfume;
(ii) at least one ingredient selected from the group consisting of a perfumery carrier and a perfuming co-ingredient; and
(iii) optionally a perfumery adjuvant.

14. A liquid consumer product, preferably in the form of a laundry care product, a home care product, a body care product, a skin care product, an air care product, or a hygiene product, said consumer product comprising:
a) from 2 to 65% by weight, relative to the total weight of the consumer product, of at least one surfactant;
b) water or a water-miscible hydrophilic organic solvent; and
c) microcapsules as defined in claim 10. defined in claim 13.

15. A powder consumer product , preferably in the form of a laundry care product, a home care product, a body care product, a skin care product, an air care product, or a hygiene product, said consumer product comprising:
(a) from 2 to 65% by weight, relative to the total weight of the consumer product, of at least one surfactant; and
(b) microcapsules as defined in claim 12.
